(19) United States
(12) Patent Application Publication
(10) Pub. No.: US 2020/0376035 A1
(43) Pub. Date: Dec. 3, 2020

(54) METHODS AND COMPOSITIONS COMPRISING CART AND A SMAC MIMETIC

(71) Applicant: THE TRUSTEES OF THE UNIVERSITY OF PENNSYLVANIA, Philadelphia, PA (US)

(72) Inventors: Marco Ruella, Ardmore, PA (US); Yong Gu Lee, Philadelphia, PA (US)

(21) Appl. No.: 16/971,995

(22) PCT Filed: Feb. 22, 2019
(86) PCT No.: PCT/US2019/019158

§ 371 Date: Aug. 21, 2020

Related U.S. Application Data
(60) Provisional application No. 62/635,377, filed on Feb. 26, 2018.

(51) Int. Cl. A61K 35/17; C07K 14/725

(52) U.S. Cl. CPC A61K 35/17; C07K 2319/33; C07K 2319/03; C07K 14/7051

(57) ABSTRACT

The present invention relates to compositions and methods comprising a T cell genetically modified to express a CAR and a SMAC mimetic for treating a patient having a disease, a disorder or a condition associated with an elevated expression of an antigen. In some embodiments, the antigen is a tumor antigen.

### CROSS-REFERENCE TO RELATED APPLICATION

The present application is entitled to priority under 35 U.S.C. § 119(e) to U.S. Provisional Patent Application No. 62/635,377 filed Feb. 26, 2018, which is hereby incorporated by reference in its entirety herein.

### STATEMENT REGARDING FEDERALLY SPONSORED RESEARCH OR DEVELOPMENT

This invention was made with government support under Grant No. 5K99CA212302-02 awarded by the National Institutes of Health. The government has certain rights in the invention.

### BACKGROUND OF THE INVENTION

Adoptive cell transfer (ACT) using chimeric antigen receptor modified T cells (CARTs) has been shown to be a promising strategy for the treatment of cancers (Louis et al., 2011, Blood 118:6050-6056; Kochenderfer et al., 2010, Blood 116:3875-3886; Porter et al., 2011, N Engl J Med 365:725-733, Maude et al., 2018, N Engl J Med 378:439-448; Schuster et al., 2017, N Engl J Med 377:2545-2554 and Porter et al., 2015, Sci Transl Med 303:303ra139). However, some disorders or cancers remain refractory to CAR treatment, and in some patients the efficacy of treatment may decrease over time.

There remains a need for methods and compositions for increasing or modulating the activity of CARs.

### SUMMARY

Provided is a composition comprising: a T cell genetically modified to express a CAR; and a SMAC mimetic. In some embodiments, the CAR comprises an antigen binding domain, a transmembrane domain, and an intracellular signaling domain. In further embodiments, the intracellular signaling domain comprises a costimulatory signaling region. In yet further embodiments, the costimulatory signaling region comprises the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83.

In some embodiments, the intracellular signaling domain comprises a CD3zeta chain.

In some embodiments, the SMAC mimetic is birinapant, (methylamino)propanamide, LCL161, GDC-0917, HGS1029, AT-406, BV-6, GDC-0152, or AZD5582, or any combinations thereof, or a salt or solvate thereof. In further embodiments, the SMAC mimetic is birinapant, or a salt or solvate thereof.

In some embodiments, the antigen is a tumor antigen. In further embodiments, the tumor antigen is selected from the group consisting of CD19, CD20, CD22, BCMA, ROR1, Mesothelin, CD33/IL3Ra, c-Met, PSMA, Glycolipid F77, EGFRvIII, GD-2, NY-ESO-1 TCR, MAGE A3 TCR and HER-2.

In some embodiments, the composition further comprises a pharmaceutically acceptable carrier or adjuvant.

Provided is a method for treating a patient having a disease, a disorder or a condition associated with an elevated expression of an antigen, the method comprising administering to the patient an effective amount of the composition of any one of the previous embodiments.

Provided is a method for treating a patient having a disease, a disorder or a condition associated with an elevated expression of an antigen, the method comprising administering to the patient an effective amount of: a T cell genetically modified to express a CAR; and a SMAC mimetic. In some embodiments, the CAR comprises an antigen binding domain, a transmembrane domain, and an intracellular signaling domain. In further embodiments, the intracellular signaling domain comprises a costimulatory signaling region. In yet further embodiments, the costimulatory signaling region comprises the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83.

In some embodiments, the intracellular signaling domain comprises a CD3zeta chain.

In some embodiments, the SMAC mimetic is selected from the group consisting of AZD5582, birinapant, LCL161, GDC-0152, GDC-0917, HGS1029, AT-406, or BV-6, any salt or solvate thereof, and any combinations thereof. In further embodiments, the SMAC mimetic is birinapant, or a salt or solvate thereof.

In some embodiments, the T cell genetically modified to express a CAR and the SMAC mimetic are administered to the patient simultaneously or sequentially.

In some embodiments, the antigen is a tumor antigen. In further embodiments, the tumor antigen is selected from the group consisting of CD19, CD20, CD22, BCMA, ROR1, Mesothelin, CD33/IL3Ra, c-Met, PSMA, Glycolipid F77, EGFRvIII, GD-2, NY-ESO-1 TCR, MAGE A3 TCR and HER-2.

In some embodiments, administration of the T cell genetically modified to express a CAR and the SMAC mimetic induces apoptosis of a cell expressing the antigen.

In some embodiments, the T cell genetically modified to express a CAR and/or the SMAC mimetic further comprises a pharmaceutically acceptable carrier or adjuvant.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1B are a series of images illustrating that birinapant enhances CART killing of leukemic cell lines. FIG. 1A is a graph showing the percentage of tumor killing by CART as a result of increasing concentrations of birinapant or corresponding amounts of DMSO as a control. FIG. 1B is a graph showing the percentage of tumor killing by CART as a function of CART:Tumor ratio. For FIGS. 1A-1B, a human pre-B ALL cell line expressing endogenous CD19 and a luciferase reporter (NALM6 CBG-T2A-GFP) was cultured in the presence or absence of human T cells expressing a chimeric antigen receptor against CD19 (CTL019, KYMRIAH®) at different ratios. Cells were treated with different doses of birinapant (TL32711, Medivir) or corresponding amounts of DMSO. After 72 hours luciferin was added to the cells and luminescence was detected using a luminometer (Biotek Synergy H4). Tumor killing was calculated using the formula: (sample−tumor treated with DMSO)/(lysis control−tumor treated with DMSO). Negative values indicate increased tumor growth.

FIGS. 2A-2D are a series of images illustrating that birinapant enhances CART killing of solid tumor cell lines. FIG. 2A is a graph showing the growth and clearance of tumor cells over time. At approximately 21 h (vertical line) tumor cells were treated with only Birinapant (solid lines) or Birinapant and CART cells (dotted lines, 1:1 CART:Tumor ratio). FIG. 2B is analogous to FIG. 2A except for the addition of DMSO instead of Birinapant. FIGS. 2C and 2D summarize the results of FIGS. 2A and 2B, respectively (10 nM Birinapant or DMSO). For FIGS. 2A-2D, a human ovarian adenocarcinoma cell line (SKOV3) expressing endogenous HER2 was cultured in the presence or absence of human T cells expressing a chimeric antigen receptor against HER2 (CAR 4D5) at a 1:1 CART:Tumor ratio, Cells were treated with different doses of birinapant (TL32711, Medivir) or corresponding amounts of DMSO. Tumor killing was monitored in real-time using the impedance-based XCelligence® system (ACEA Biosciences, Inc),

FIG. 3 illustrates that birinapant-enhanced CART killing of tumor is TNF-dependent. A human pre-B ALL cell line expressing endogenous CD19 and a luciferase reporter (NALM6 CBG-T2A-GFP) was cultured in the presence or absence of human T cells expressing a chimeric antigen receptor against CD19 (CTL019, KYMRIAH®). Cells were either treated with 1 μM of birinapant (TL32711, Medivir), corresponding amounts of DMSO or 1 μM birinapant (TL32711, Medivir) in combination with different amounts of isotype control or TNF antibodies. After 72 hours, luciferin was added to the cells and luminescence was detected using a luminometer (Biotek Synergy H4), Tumor killing was calculated using the formula: (sample−tumor treated with DMSO)/(lysis control−tumor treated with DMSO).

FIG. 4 summarizes the results shown in FIG. 3.

FIG. 5 illustrates the TNF apoptosis signaling pathway and illustrates how birinapant (or SMAC) can re-establish the TNF apoptosis signal by binding to cIAP1, an inhibitor of apoptosis protein.

FIG. 6 illustrates a high-throughput platform to identify CART-enhancing bioactive compounds. The CD19+ Luc+ NALM-6 leukemia cell line was plated at 0.3:1 E:T ratio+/− CART19 using an automated dispenser, Small molecules from a custom-made library (SelleckChem) were then added via an automated workstation and luminescence was detected after 48 hours with a high-throughput plate reader.

FIG. 7 illustrates that birinapant enhances CART cell cytotoxicity. Screening results: total cytotoxicity (%) of small molecules alone (Y axis) or in combination with CART (X axis). Birinapant is represented by the dot at the “40” mark on the X axis (●).

FIG. 8 illustrates a high-throughput platform to identify CART-enhancing bioactive compounds. The CD19+ Luc+ NALM-6 leukemia cell line was plated at 0.3:1 E:T ratio+/− CART19 using an automated dispenser. Small molecules from a custom-made library (SelleckChem) were then added via an automated workstation and luminescence was detected after 48 hours with a high-throughput plate reader,

FIGS. 9A-9B illustrate the identification of potent small molecule candidates that improve CAR T cell anti-tumor efficacy. FIG. 9A shows 1000 nM of small molecule libraries tested to identify the potent combination of small molecule and CART19. Screening results: total cytotoxicity (%) of small molecules alone (Y axis) or in combination with CART (X axis). FIG. 9B shows 100 nM of small molecule libraries tested to identify the potent combination of small molecule and CART19. Screening results: total cytotoxicity (c/o) of small molecules alone (Y axis) or in combination with CART (X axis).

FIG. 10 illustrates commercially available SMAC mimetics.

FIGS. 11A-11B illustrate that SMAC mimetics enhance the anti-tumor efficacy of a CAR T cell against a B-cell leukemia cell line (NALM6). FIG. 11A shows the effect of SMAC mimetics on tumor killing with various doses of SMAC mimetics (10, 50, 100, 500 and 1000 μnM) introduced in the absence of CART19. FIG. 11B shows the effect of SMAC mimetics on tumor killing with various doses of SMAC mimetics (10, 50, 100, 500 and 1000 nM) introduced in the presence of CART19. Tumor killing was quantified after a 48 hour co-culture by measuring the change of luminescent activity. E:T ratio=0.03:1.

FIGS. 12A-12B illustrate the synergistic killing of solid tumors using CAR cells in combination with SMAC mimetics. FIG. 12A shows anti-HER2 CAR T (4D5) cell co-cultured with a HER2+ human ovarian cancer cell line (SKOV3) for 96 hours with or without birinapant (50 nM). FIG. 12B shows a comparison of the synergistic effect of top 3 SMAC mimetics with CAR T cells against SKOV3. E:T ratio=0.01:1, [Drug]=50 nM. 24 h co-culture,

FIG. 13 illustrates that SMAC mimetics enhance tumor killing of both CD28 and 4-1BB costimulated CAR T cells. T cells with either a CD28− (See Milone et al. Mol Ther 2009; 17(8):1453-64, only for CD28 feature of the CAR) or 4-1BB-based CAR19 (CTL019) were co-cultured with B-ALL cells (NALM6) in the presence of the top 3 SMAC mimetics or vehicle. After 48 hours, total tumor killing was recorded by measuring the change of luminescent activity in each well. ET ratio=0.03:1.

FIGS. 14A-14E illustrate that SMAC mimetics in combination with CAR T cells induce apoptosis of tumor cells. CART 19s were co-cultured with NALM6 in the presence of top 3 SMAC mimetics. The change of caspase activity in cancer cells was monitored after 24 h co-culture with CART19 and SMAC mimetics via flow cytometry analysis. E:T ratio=0.27:1. [Drug]=1 μM. FIG. 14A shows CART19 cells alone. FIG. 14B shows CART19 cultured with DMSO. FIG. 14C shows CART19 co-cultured with Birinapant (1 uM). FIG. 14D shows CART19 co-cultured with BV6 (1 μM). FIG. 14E shows CART19 co-cultured with AZD5582 (1 μM).

FIG. 15 illustrates the inhibition of caspase activity drastically reduces the synergy between CART cells and SMAC mimetics. CART19 cells were co-cultured with NALM6 (CBG-T2A-GFP) in the presence of DMSO (1 μM), birinapant (1 μM), BV6 (1 μM) or AZD5582 (1 μM) with or without the pan-caspase inhibitor (Z-VAD-FMK (20 μM)). After 48 hours, total tumor killing was monitored by measuring the change of luminescence intensity, E:T ratio=0.03:1.

FIGS. 16A-16B illustrate the synergy between CAR T cells and SMAC mimetics is mediated by TNFα. FIG. 16A shows the synergy between CAR T cell and SMAC mimetics [(DMSO (1 μM), birinapant (1 μM) or BV6 (1 μM)], neutralizing antibodies for death ligands: TNFa (1 μg/ml), TRAIL (1 μg/ml), FasL (1 μg/ml). NALM6 (CBG-T2A-GFP) killing was measured by monitoring change of luminescent activity after 48 hours. E:T ratio=0.03:1. FIG. 16B shows the synergy between CART cell and SMAC mimetics [(DMSO (1 μg/ml), birinapant (1 μM) or BV6 (1 μM)], neutralizing antibodies for TNF receptor 1 (TNFR1). NALM6 (CBG-T2A-GFP) killing was measured by monitoring change of luminescent activity after 48 hours. E:T ratio=0.03:1.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa (κ) and lambda (λ) light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “auto-antigen” means, in accordance with the present invention, any self-antigen which is recognized by the immune system as being foreign. Auto-antigens comprise, but are not limited to, cellular proteins, phosphoproteins, cellular surface proteins, cellular lipids, nucleic acids, glycoproteins, including cell surface receptors.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have been expressed with specificity to a tumor associated antigen, for example. CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies, fused to CD3zeta transmembrane and intracellular domain. The specificity of CAR designs may be derived from ligands of receptors (e.g., peptides). In some embodiments, a CAR can target cancers by redirecting the specificity of a T cell expressing the CAR specific for tumor associated antigens.

The term “cleavage” refers to the breakage of covalent bonds, such as in the backbone of a nucleic acid molecule. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

The term “CRISPR/CAS,” “clustered regularly interspaced short palindromic repeats system,” or “CRISPR” refers to DNA loci containing short repetitions of base sequences. Each repetition is followed by short segments of spacer DNA from previous exposures to a virus. Bacteria and archaea have evolved adaptive immune defenses termed CRISPR-CRISPR-associated (Cas) systems that use short RNA to direct degradation of foreign nucleic acids. In bacteria, the CRISPR system provides acquired immunity against invading foreign DNA via RNA-guided DNA cleavage.

In the type II CRISPR/Cas system, short segments of foreign DNA, termed “spacers” are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Recent work has shown that target recognition by the Cas9 protein requires a “seed” sequence within the crRNA and a conserved dinucleotide-containing protospacer adjacent motif (PAM) sequence upstream of the crRNA-binding region.

To direct Cas9 to cleave sequences of interest, crRNA-tracrRNA fusion transcripts, hereafter referred to as “guide RNAs” or “gRNAs” may be designed, from human U6 polymerase III promoter. CRISPR/CAS mediated genome editing and regulation, highlighted its transformative potential for basic science, cellular engineering and therapeutics.

The term “CRISPRi” refers to a CRISPR system for sequence specific gene repression or inhibition of gene expression, such as at the transcriptional level.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

As used here, “induced pluripotent stem cell” or “iPS cell” refers to a pluripotent stem cell that is generated from adult cells, such as T cells. The expression of reprogramming factors, such as Klf4, Oct3/4 and Sox2, in adult cells convert the cells into pluripotent cells capable of propagation and differentiation into multiple cell types.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “Sendai virus” refers to a genus of the Paramyxoviridae family. Sendai viruses are negative, single stranded RNA viruses that do not integrate into the host genome or alter the genetic information of the host cell. Sendai viruses have an exceptionally broad host range and are not pathogenic to humans. Used as a recombinant viral vector, Sendai viruses are capable of transient but strong gene expression.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

The term “SMAC mimetic” as used herein, refers to a drug or compound that mimics the second mitochondria-derived activator of caspases (SMAC), a pro-apoptotic mitochondrial protein that is an endogenous inhibitor of a family of cellular proteins known as inhibitor of apoptosis proteins (IAPs).

Non-limiting examples of a SMAC mimetic, or a salt or solvate thereof, comprise:

AZD5582 (also known as (2S,2′S)-N,N′-((S,1′S,2R,2′R)-(hexa-2,4-diyne-1,6-diylbis(oxy))bis(2,3-dihydro-1H-indene-2,1-diyl))bis(1-((S)-2-cyclohexyl-2-((S)-2-(methylamino)propanamido)acetyl)pyrrolidine-2-carboxamide)):

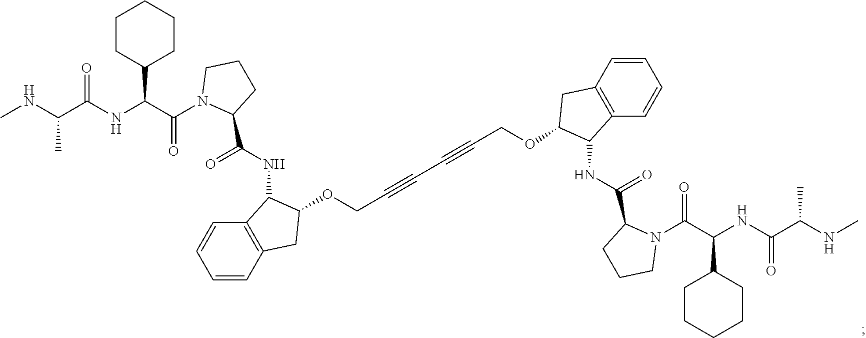

birinapant (also known as TL-32711, or (2S)-N-[(2S)-1-[(2R,4S)-2-[[6-fluoro-2-[6-fluoro-3-[[(2R,4S)-4-hydroxy-1-[(2S)-2-[[(2S)-2-(methylamino)propanoyl]amino]butanoyl]pyrrolidin-2-yl]methyl]-1H-indol-2-yl]-1H-indol-3-yl]methyl]-4-hydroxypyrrolidin-1-yl]-1-oxobutan-2-yl]-2-(methylamino)propanamide, TetraLogic Pharmaceuticals):

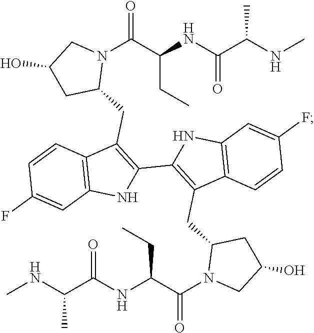 [SMILES: CC[C@H](NC(=O)[C@H](C)NC)C(=O)N1C[C@@H](O)C[C@H]1Cc1c(-c2[nH]c3cc(F)ccc3c2C[C@@H]2C[C@H](O)CN2C(=O)[C@H](CC)NC(=O)[C@H](C)NC)[nH]c2cc(F)ccc12]

LCL161 ((S)-N-((S)-1-cyclohexyl-2-((S)-2-(4-(4-fluorobenzoyl)thiazol-2-yl)pyrrolidin-1-yl)-2-oxoethyl)-2-(methylamino)propanamide; Novartis):

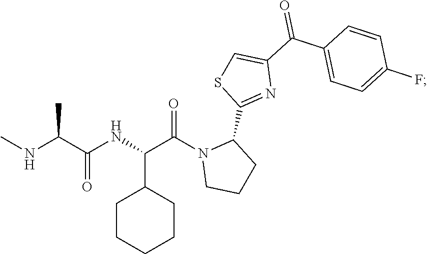 [SMILES: CN[C@@H](C)C(=O)N[C@H](C(=O)N1CCC[C@H]1c1nc(C(=O)c2ccc(F)cc2)cs1)C1CCCCC1]

GDC-0152 ((S)-1-((S)-2-cyclohexyl-2-((S)-2-(methylamino)propanamido)acetyl)-N-(4-phenyl-1,2,3-thiadiazol-5-yl)pyrrolidine-2-carboxamide, Genentech):

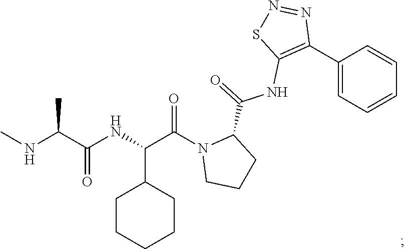 [SMILES: CN[C@@H](C)C(=O)N[C@H](C(=O)N1CCC[C@H]1C(=O)Nc1snnc1-c1ccccc1)C1CCCCC1]

GDC-0917 ((S)-1-((S)-2-cyclohexyl-2-((S)-2-(methylamino)propanamido)acetyl)-N-(2-(oxazol-2-yl)-4-phenylthiazol-5-yl)pyrrolidine-2-carboxamide, Genentech):

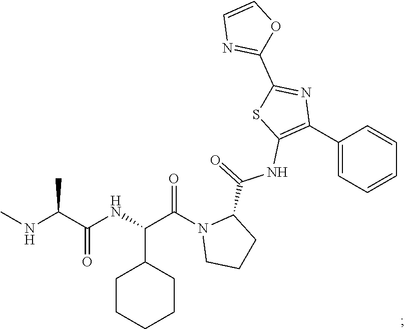 [SMILES: CN[C@@H](C)C(=O)N[C@H](C(=O)N1CCC[C@H]1C(=O)Nc1sc(-c2ncco2)nc1-c1ccccc1)C1CCCCC1]

HGS1029 (Human Genome Sciences), and AT-406 ((5S,8S,10aR)-N-(diphenylmethyl)decahydro-5-[[(2S)-2-(methylamino)-1-oxopropyl]amino]-3-(3-methyl-1-oxobutyl)-6-oxo-pyrrolo[1,2-a][1,5]diazocine-8-carboxamide, Ascenta):

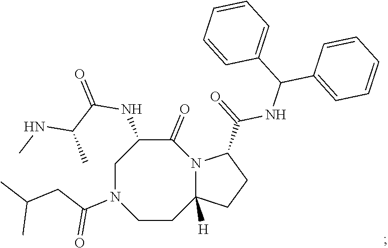 [SMILES: CN[C@@H](C)C(=O)N[C@H]1CN(C(=O)CC(C)C)CC[C@H]2CC[C@@H](C(=O)NC(c3ccccc3)c3ccccc3)N2C1=O]

BV-6 (also known as BV6, or ((S,S,2S,2′S)-N,N′-((2S,2′S)-(hexane-1,6-diylbis(azanediyl)) bis(3-oxo-1,1-diphenylpropane-3,2-diyl))bis(1-((S)-2-cyclohexyl-2-((S)-2-(methylamino)propanamido)acetyl)pyrrolidine-2-carboxamide)), Genentech):

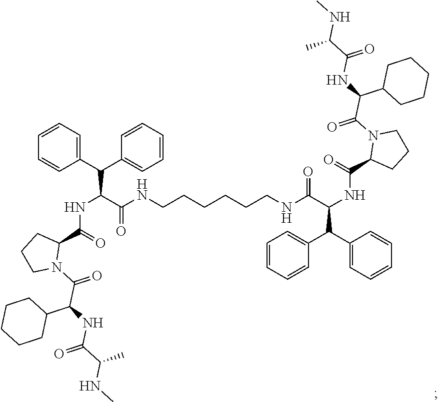 [SMILES: CN[C@@H](C)C(=O)N[C@H](C(=O)N1CCC[C@H]1C(=O)N[C@H](C(=O)NCCCCCCNC(=O)[C@@H](NC(=O)[C@@H]1CCCN1C(=O)[C@@H](NC(=O)[C@H](C)NC)C1CCCCC1)C(c1ccccc1)c1ccccc1)C(c1ccccc1)c1ccccc1)C1CCCCC1]

or a salt or solvate thereof.

The term “Inhibitors of Apoptosis Proteins (IAPs)” as used herein, refers to a family of functionally and structurally related proteins that serve as endogenous inhibitors of programmed cell death or apoptosis. All IAPs contain one to three copies of a BIR (Baculovirus IAP Repeat), a domain comprising about 70 amino acids.

The term “second mitochondria-derived activator of caspases (SMAC)” as used herein refers to a mitochondrial protein also referred to as DIABLO, that binds IAPs, thus freeing caspases to activate apoptosis.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (P) chain, although in some cells the TCR consists of gamma and delta (y/S) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

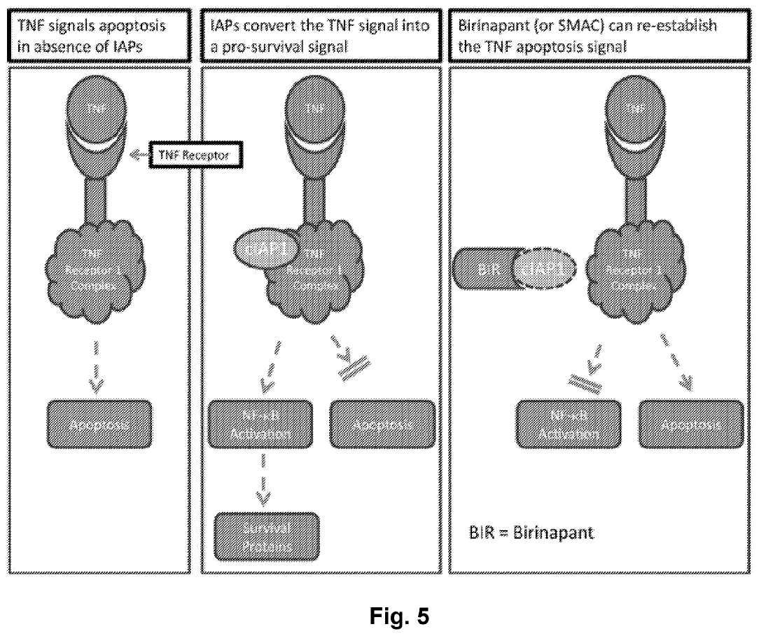

The invention relates to compositions and methods for treating a disease. In some embodiments, the disease is a cancer, including but not limited to hematologic malignancies and solid tumors. The invention also encompasses methods of treating and preventing a disease, such as certain types of cancer, including primary and metastatic cancer, as well as cancers that are refractory or resistant to conventional chemotherapy. The methods comprise administering to a patient in need of such treatment or prevention a therapeutically or prophylactically effective amount of a T cell transduced to express a chimeric antigen receptor (CAR). Provided are compositions and methods comprising a CAR and a SMAC mimetic. CARs are molecules that combine antibody-based specificity for a desired antigen (e.g., tumor antigen) with a T cell receptor-activating intracellular domain to generate a chimeric protein that exhibits a specific anti-tumor cellular immune activity. SMAC mimetics are drugs or compounds that mimic the second mitochondria derived activator of caspase (SMAC), a pro-apoptotic mitochondrial protein that is an endogenous inhibitor of a family of cellular proteins known as the Inhibitor of Apoptosis Proteins (IAPs). In some embodiments, the SMAC mimetic is AZD5582, birinapant, LCL161, GDC-0152, GDC-0917, HGS1029, AT-406, or BV-6, or any combinations thereof, or a salt or solvate thereof. SMAC mimetics such as birinapant suppresses IAPs, and thus induce apoptosis as illustrated in FIG. 5.

In some embodiments of any of the methods above, the methods result in a measurable reduction in tumor size or evidence of disease or disease progression, complete response, partial response, stable disease, increase or elongation of progression free survival, increase or elongation of overall survival, or reduction in toxicity.

In one embodiment, the CAR of the invention comprises an extracellular domain having an antigen recognition domain that targets a desired antigen, a transmembrane domain, and a cytoplasmic domain.

In one embodiment, the CAR of the invention can be engineered to comprise an extracellular domain having an antigen binding domain that targets tumor antigen fused to an intracellular signaling domain of the T cell antigen receptor complex zeta chain (e.g., CD3zeta). An exemplary tumor antigen B cell antigen is CD19 because this antigen is expressed on malignant B cells. However, the invention is not limited to targeting CD19. Rather, the invention includes any tumor antigen binding moiety. The antigen binding moiety is preferably fused with an intracellular domain from one or more of a costimulatory molecule and a zeta chain. Preferably, the antigen binding moiety is fused with one or more intracellular domains selected from the group of a CD137 (4-1BB) signaling domain, a CD28 signaling domain, a CD3zeta signal domain, and any combination thereof.

In one embodiment, the CAR of the invention comprises a CD137 (4-1BB) signaling domain. This is because the present invention is partly based on the discovery that CAR-mediated T-cell responses can be further enhanced with the addition of costimulatory domains. For example, inclusion of the CD137 (4-1BB) signaling domain significantly increased CAR mediated activity and in vivo persistence of CAR T cells compared to an otherwise identical CAR T cell not engineered to express CD137 (4-1BB). However, the invention is not limited to a specific CAR. Rather, any CAR that targets a desired antigen, for example a tumor antigen, can be used in the present invention. Compositions and methods of making and using CARs have been described in PCT/US11/64191, which is incorporated by reference in its entirety herein.

Chimeric Antigen Receptors

The present invention provides a T cell genetically modified to express a chimeric antigen receptor (CAR) comprising an extracellular and intracellular domain. Compositions and methods of making CARs have been described in PCT/US11/64191, which is incorporated in its entirety by reference herein.

The extracellular domain comprises a target-specific binding element otherwise referred to as an antigen binding domain. In some embodiments, the extracellular domain also comprises a hinge domain. In one embodiment, the intracellular domain or otherwise the cytoplasmic domain comprises a costimulatory signaling region and a zeta chain portion. The costimulatory signaling region refers to a portion of the CAR comprising the intracellular domain of a costimulatory molecule. Costimulatory molecules are cell surface molecules other than antigen receptors or their ligands that are required for an efficient response of lymphocytes to antigen.

Between the extracellular domain and the transmembrane domain of the CAR, or between the cytoplasmic domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

The present invention includes retroviral and lentiviral vector constructs expressing a CAR that can be directly transduced into a cell. The present invention also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the gene to be expressed, and a polyA tail, typically 50-2000 bases in length. RNA so produced can efficiently transfect different kinds of cells. In one embodiment, the template includes sequences for the CAR.

Preferably, the CAR comprises an extracellular domain, a transmembrane domain and a cytoplasmic domain. The extracellular domain and transmembrane domain can be derived from any desired source of such domains. In some instances, the hinge domain of the CAR of the invention comprises the CD8a hinge domain.

In one embodiment, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus examples of cell surface markers that may act as ligands for the antigen moiety domain in the CAR of the invention include those associated with viral, bacterial and parasitic infections, autoimmune disease and cancer cells.

In one embodiment, the CAR of the invention can be engineered to target a tumor antigen of interest by way of engineering a desired antigen binding domain that specifically binds to an antigen on a tumor cell. In the context of the present invention, “tumor antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those of skill in the art.

Tumor antigens are proteins that are produced by tumor cells that elicit an immune response, particularly T-cell mediated immune responses. The selection of the antigen binding domain of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

In some embodiments, the antigen is CD22, CD123, CD33, CD79b, ROR-1, CAIX, mesothelin, CMET, CD70, CLL-1, IL1RA, CD38, BCMA, CS-1, MUC1, CD2, CD5, CD7, CD30, CCR4, CD4, CD8, CD3, CD37, NKIG2D or FLT-3.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3CA 27.29BCAA, CA 195, CA 242, CA-50, CAM43, CD68P1, CO-029, FGF-5, G250, Ga733EpCAM, HTgp-175, M344, MA-50, MG7-Ag, MOV18, NB/70K, NY-CO-1, RCAS1, SDCCAG16, TA-90Mac-2 binding protein\cyclophilin C-associated protein, TAAL6, TAG72, TLP, and TPS.

In a preferred embodiment, the antigen binding domain portion of the CAR targets an antigen that includes but is not limited to CD19, CD20, CD22, BCMA, ROR1, Mesothelin, CD33/IL3Ra, c-Met, PSMA, Glycolipid F77, EGFRvIII, GD-2, NY-ESO-1 TCR, MAGE A3 TCR, HER-2 and the like.

Depending on the desired antigen to be targeted, the CAR of the invention can be engineered to include the appropriate antigen bind moiety that is specific to the desired antigen target.

The antigen binding domain can be any domain that binds to the antigen including but not limited to monoclonal antibodies, polyclonal antibodies, synthetic antibodies, human antibodies, humanized antibodies, and fragments thereof. In some instances, it is beneficial for the antigen binding domain to be derived from the same species in which the CAR will ultimately be used in. For example, for use in humans, it may be beneficial for the antigen binding domain of the CAR to comprise a human antibody or fragment thereof. Thus, in one embodiment, the antigen biding domain portion comprises a human antibody or a fragment thereof. Alternatively, in some embodiments, a non-human antibody is humanized, where specific sequences or regions of the antibody are modified to increase similarity to an antibody naturally produced in a human.

In one embodiment of the present invention, a plurality of types of CARs is expressed on the surface of a T cell. The different types of CAR may differ in their antigen binding domain. That is, in one embodiment, the different types of CARs each bind a different antigen. In one embodiment, the different antigens are markers for a specific tumor. For example, in one embodiment, the different types of CARs each bind to a different antigen, where each antigen is expressed on a specific type of tumor. Examples of such antigens are discussed elsewhere herein.

With respect to the transmembrane domain, the CAR can be designed to comprise a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, ICOS. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

The cytoplasmic domain or otherwise the intracellular signaling domain of the CAR of the invention is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In one embodiment of the present invention, the effector function of the cell is dependent upon the binding of a plurality of types of CARs to their targeted antigen. For example, in one embodiment, binding of one type of CAR to its target is not sufficient to induce the effector function of the cell.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3zeta.

In one embodiment, the cytoplasmic domain of the CAR can be designed to comprise the CD3zeta signaling domain by itself or combined with any other desired cytoplasmic domain(s) useful in the context of the CAR of the invention. For example, the cytoplasmic domain of the CAR can comprise a CD3zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

The cytoplasmic signaling sequences within the cytoplasmic signaling portion of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain is designed to comprise the signaling domain of CD3zeta. In another embodiment, the cytoplasmic domain is designed to comprise the signaling domain of CD3zeta and the signaling domain of 4-1BB. In one embodiment of the present invention, a plurality of types of CARs is expressed on a cell, where the different types of CAR may vary in their cytoplasmic domain. In one embodiment, at least one type of CAR comprises the CD3zeta domain, while at least one type of CAR comprises a costimulatory domain, for example the 4-1BB domain. However, the different types of CARs are not limited by any particular cytoplasmic domain. For example, each type of CAR can comprise any ITAM containing sequence, costimulatory domain, or combination thereof such that binding of each individual type of CAR is insufficient to induce effector function but binding of a plurality of types of CARs are able to induce effector function. That is, the domains of each type of CAR work together to induce effector function.

In one embodiment, the T cell genetically modified to express a CAR is further engineered or edited using CRISPR/Cas.

Extracellular Domain

The present invention includes an extracellular domain comprising an antigen binding domain derived from an antibody directed against a co-stimulatory molecule. The co-stimulatory molecule can include any molecule that co-stimulates T cells, such as, but not limited to, CD3, CD28, or a combination thereof. In one embodiment, the extracellular domain can include an antigen binding domain derived from anti-CD3, anti-CD28, or a combination thereof.

In another embodiment, the extracellular domain can include any portion of an antibody that binds to antigen including, but not limited to, the antigen binding domain of a synthetic antibody, human antibody, humanized antibody, single domain antibody, single chain variable fragments, and fragments thereof. In some instances, it is beneficial for the extracellular domain to be derived from the same species in which the chimeric membrane protein will ultimately be used in. For example, for use in humans, it may be beneficial for the extracellular domain of the chimeric membrane protein to comprise a human antibody or fragment thereof. Thus, in one embodiment, the extracellular domain portion comprises a human antibody or a fragment thereof.

For in vivo use of antibodies in humans, it may be preferable to use human antibodies. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety. A human antibody can also be an antibody wherein the heavy and light chains are encoded by a nucleotide sequence derived from one or more sources of human DNA.

Alternatively, in some embodiments, a non-human antibody is humanized, where specific sequences or regions of the antibody are modified to increase similarity to an antibody naturally produced in a human. In one embodiment, the antigen binding domain portion is humanized.

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some FR residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carteret al., Proc. Natl. Acad. Sci. USA, 89:4285 (1992); Presta et al., J. Immunol., 151:2623 (1993), the contents of which are incorporated herein by reference herein in their entirety).

Antibodies can be humanized with retention of high affinity for the target antigen and other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

A “humanized” antibody retains a similar antigenic specificity as the original antibody. However, using certain methods of humanization, the affinity and/or specificity of binding of the antibody for human CD3 antigen may be increased using methods of “directed evolution,” as described by Wu et al., J. Mol. Biol., 294:151 (1999), the contents of which are incorporated herein by reference herein in their entirety.

In one embodiment, the antibody is a synthetic antibody, human antibody, a humanized antibody, single chain variable fragment, single domain antibody, an antigen binding fragment thereof, and any combination thereof.

Intracellular Domain

The intracellular domain or cytoplasmic domain comprises a costimulatory signaling region. The costimulatory signaling region refers to an intracellular domain of a costimulatory molecule. Costimulatory molecules are cell surface molecules other than antigen receptors or their ligands that are required for an efficient response of lymphocytes to antigen.

The cytoplasmic domain or the intracellular signaling domain of the chimeric membrane protein is responsible for activation of at least one of effector functions of the T cell. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Nonlimiting examples of intracellular signaling domains for use in the chimeric membrane protein include any portion of the intracellular domain of CD28, 4-1BB, T cell receptor (TCR), co-stimulatory molecules, any derivative or variant of these sequences, any synthetic sequence that has the same functional capability, and any combination thereof.

Other Domains of the Chimeric Membrane Protein

Between the extracellular domain and the transmembrane domain of the chimeric membrane protein, or between the cytoplasmic domain and the transmembrane domain of the chimeric membrane protein, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

In some embodiments, the chimeric membrane protein further comprises a transmembrane domain. In some embodiment, the chimeric membrane protein further comprises a hinge domain. In one embodiment, the RNA encoding the chimeric membrane protein further comprises a transmembrane and hinge domain, such as a CD28 transmembrane domain and a CD8alpha hinge domain.

SMAC Mimetics

The present invention relates to compositions and methods comprising a T cell genetically modified to express a CAR and a SMAC mimetic for treating a patient having a disease, a disorder or a condition associated with an elevated expression of a disease or tumor antigen. A SMAC mimetic is a drug or compound that mimics the second mitochondria derived activator of caspase (SMAC), a pro-apoptotic mitochondrial protein that is an endogenous inhibitor of a family of cellular proteins known as the Inhibitor of Apoptosis Proteins (IAPs).

An example of a SMAC mimetic is birinapant. Without wishing to be bound by theory, it is believed that SMAC mimetics such as birinapant function by binding IAPs such as cIAP1 and cIAP2, as illustrated in FIG. 5.

SMAC mimetics contemplated within the invention include:

AZD5582 (also known as (2S,2′S)-N,N′-((1S,1′S,2R,2′R)-(hexa-2,4-diyne-1,6-diylbis(oxy))bis(2,3-dihydro-1H-indene-2,1-diyl))bis(1-((S)-2-cyclohexyl-2-((S)-2-(methylamino)propanamido)acetyl)pyrrolidine-2-carboxamide));

birinapant (also known as TL-32711, or (2S)-N-[(2S)-1-[(2R,4S)-2-[[6-fluoro-2-[6-fluoro-3-[[(2R,4S)-4-hydroxy-1-[(2S)-2-[[(2S)-2-(methylamino)propanoyl]amino]butanoyl]pyrrolidin-2-yl]methyl]-1H-indol-2-yl]-1H-indol-3-yl]methyl]-4-hydroxypyrrolidin-1-yl]-1-oxobutan-2-yl]-2-(methylamino)propanamide, TetraLogic Pharmaceuticals);

LCL161 ((S)-N-((S)-1-cyclohexyl-2-((S)-2-(4-(4-fluorobenzoyl)thiazol-2-yl)pyrrolidin-1-yl)-2-oxoethyl)-2-(methylamino)propanamide; Novartis);

GDC-0152 ((S)-1-((S)-2-cyclohexyl-2-((S)-2-(methylamino)propanamido)acetyl)-N-(4-phenyl-1,2,3-thiadiazol-5-yl)pyrrolidine-2-carboxamide, Genentech);

GDC-0917 ((S)-1-((S)-2-cyclohexyl-2-((S)-2-(methylamino)propanamido)acetyl)-N-(2-(oxazol-2-yl)-4-phenylthiazol-5-yl)pyrrolidine-2-carboxamide, Genentech);

HGS1029 (Human Genome Sciences);

AT-406 ((5S,8S,10aR)-N-(diphenylmethyl)decahydro-5-[[(2S)-2-(methylamino)-1-oxopropyl]amino]-3-(3-methyl-1-oxobutyl)-6-oxo-pyrrolo[1,2-a][1,5]diazocine-8-carboxamide, Ascenta);

BV-6 (also known as BV6, or ((S,S,2S,2′S)-N,N′-((2S,2′S)-(hexane-1,6-diylbis(azanediyl)) bis(3-oxo-1,1-diphenylpropane-3,2-diyl))bis(1-((S)-2-cyclohexyl-2-((S)-2-(methylamino)propanamido)acetyl)pyrrolidine-2-carboxamide)), Genentech);

or a salt or solvate thereof. Thus, in some embodiments, the SMAC mimetic is AZD5582, birinapant, LCL161, GDC-0152, GDC-0917, HGS1029, AT-406, or BV-6, or any combinations thereof, or a salt or solvate thereof.

Therapy

The T cells genetically modified to express a CAR described herein and/or a SMAC mimetic may be included in a composition for therapy either separately or in combination. The composition may include a pharmaceutical composition and further include a pharmaceutically acceptable carrier. A therapeutically effective amount of the pharmaceutical composition comprising the T cells genetically modified to express a CAR described herein and/or a SMAC mimetic may be administered.

In some embodiments, the T cell genetically modified to express a CAR and the SMAC mimetic are administered to the patient simultaneously or sequentially. In some embodiments, the T cell genetically modified to express a CAR and the SMAC mimetic are administered to the patient in the same composition. In some embodiments, the T cell genetically modified to express a CAR and the SMAC mimetic are administered to the patient as separate compositions.

In one aspect, the invention includes a method for stimulating a T cell-mediated immune response to a target cell or tissue in a subject comprising administering to a subject an effective amount of a modified T cell. The T cells genetically modified to express a CAR described herein and/or a SMAC mimetic may be administered to induce lysis of the target cell or tissue, such as where the induced lysis is antibody-dependent cell-mediated cytotoxicity (ADCC).

In another aspect, the invention includes a method for adoptive cell transfer therapy comprising administering a population of T cells genetically modified to express a CAR described herein and/or a SMAC mimetic to a subject in need thereof to prevent or treat an immune reaction that is adverse to the subject.

In yet another embodiment, a method of treating a disease or condition associated with enhanced immunity in a subject comprising administering a population of T cells genetically modified to express a CAR described herein and/or a SMAC mimetic to a subject in need thereof.

The modified T cells generated as described herein are uniform and possess T cell function. Further, the T cells genetically modified to express a CAR described herein and/or a SMAC mimetic can be administered to an animal, preferably a mammal, even more preferably a human, to suppress an immune reaction, such as those common to autoimmune diseases such as diabetes, psoriasis, rheumatoid arthritis, multiple sclerosis, GVHD, enhancing allograft tolerance induction, transplant rejection, and the like. In addition, the cells of the present invention can be used for the treatment of any condition in which a diminished or otherwise inhibited immune response, especially a cell-mediated immune response, is desirable to treat or alleviate the disease. In one aspect, the invention includes treating a condition, such as an autoimmune disease, in a subject, comprising administering to the subject a therapeutically effective amount of a pharmaceutical composition comprising a population of modified T cells.

Examples of autoimmune disease include but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

The expanded T cells generated as described herein can also be modified and used to treat inflammatory disorders. Examples of inflammatory disorders include but are not limited to, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

In another embodiment, the T cells described herein may be used for the manufacture of a medicament for the treatment of an immune response in a subject in need thereof.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

The cells of the invention to be administered may be autologous, allogeniec or xenogenic with respect to the subject undergoing therapy.

The administration of the cells or compositions of the invention may be carried out in any convenient manner known to those of skill in the art. The cells or compositions of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

The cells or compositions described herein can also be administered using any number of matrices. The present invention utilizes such matrices within the novel context of acting as an artificial lymphoid organ to support, maintain, or modulate the immune system, typically through modulation of T cells. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. No. 5,980,889; 5,913,998; 5,902,745; 5,843,069; 5,787,900; or 5,626,561 are utilized, as such these patents are incorporated herein by reference in their entirety. Matrices comprise features commonly associated with being biocompatible when administered to a mammalian host. Matrices may be formed from natural and/or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release of seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

Pharmaceutical Compositions

Pharmaceutical compositions of the present invention may comprise a T cell genetically modified to express a CAR as described herein, and/or a SMAC mimetic, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-immune response effective amount”, “an immune response-inhibiting effective amount”, or “therapeutic amount” is indicated, the precise amount of the compositions of the present invention to be administered can be determined by a physician with consideration of individual differences in age, weight, immune response, and condition of the patient (subject). It can generally be stated that a pharmaceutical composition comprising the modified T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 ml to 400 ml. In certain embodiments, T cells are activated from blood draws of 20 ml, 30 ml, 40 ml, 50 ml, 60 ml, 70 ml, 80 ml, 90 ml, or 100 ml. Not to be bound by theory, using this multiple blood draw/multiple reinfusion protocol, may select out certain populations of T cells.

In certain embodiments of the present invention, cells expanded and modified using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

The dosage of a SMAC mimetic to be administered to a patient may be from about 0.1 to about 100 mg/m². In certain embodiments, the dosage may be from about 0.1 to about 70 mg/m². In certain embodiments, the dosage may be from about 0.1 to about 60 mg/m². In some embodiments, the dosage may be from about 0.1 to about 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m². In some embodiments, the dosage may be about 0.1, 1, 5, 10, 15, 20, 15, 30, 35, 40, 41, 42, 43, 44, 45, 46, 47, 48, 49, 50, 51, 52, 53, 54, 55, 56, 57, 58, 59 or 60 mg/m².

Human dosage amounts can initially be determined by extrapolating from the amount of compound used in mice, as a skilled artisan recognizes it is routine in the art to modify the dosage for humans compared to animal models. In certain embodiments it is envisioned that the dosage may include an effective amount from between about 0.001 mg compound/Kg body weight to about 100 mg compound/Kg body weight; or from about 0.05 mg/Kg body weight to about 75 mg/Kg body weight or from about 0.1 mg/Kg body weight to about 50 mg/Kg body weight; or from about 0.5 mg/Kg body weight to about 40 mg/Kg body weight; or from about 0.1 mg/Kg body weight to about 30 mg/Kg body weight; or from about 1 mg/Kg body weight to about 20 mg/Kg body weight. In other embodiments, the effective amount may be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, or about 100 mg/Kg body weight. In other embodiments, it is envisaged that effective amounts may be in the range of about 2 mg compound to about 100 mg compound. In other embodiments, the effective amount may be about 1, 2, 3, 4, 5, 6, 7, 8, 9, 10, 11, 12, 13, 14, 15, 16, 17, 18, 19, or 20 mg per single dose. In another embodiment, the effective amount comprises less than about 2, 3, 4, 5, 6, 7, 8, 9, 10, 15, 20, 25, 30, 35, 40, 45, 50, 55, 60, 65, 70, 75, 80, 85, 90, 95 mg daily. In an exemplary embodiment, the effective amount comprises less than about 50 mg daily. Of course, the single dosage amount or daily dosage amount may be adjusted upward or downward, as is routinely done in such treatment protocols, depending on the results of the initial clinical trials and the needs of a particular patient.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration of a composition of the invention to a suitable animal model to identify the dosage of the composition(s), concentration of components therein and timing of administering the composition(s), which elicit tissue repair, reduce cell death, or induce another desirable biological response. Such determinations do not require undue experimentation, but are routine and can be ascertained without undue experimentation.

The biologically active agents can be conveniently provided to a subject as sterile liquid preparations, e.g., isotonic aqueous solutions, suspensions, emulsions, dispersions, or viscous compositions, which may be buffered to a selected pH. Cells and agents of the invention may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by suspending talampanel and/or perampanel in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1—Screen of Compound Library for Compounds that Enhance CART Killing of Tumor

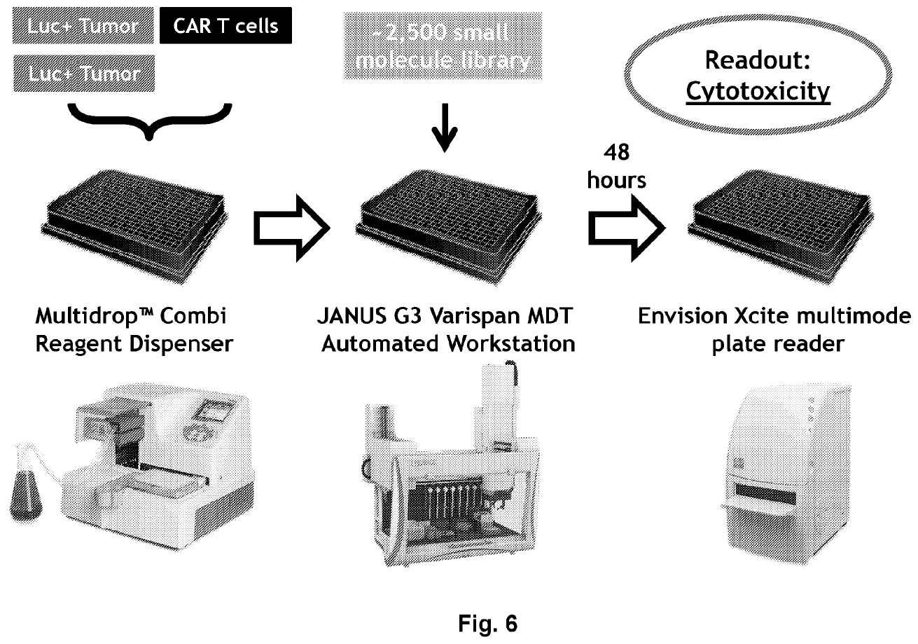

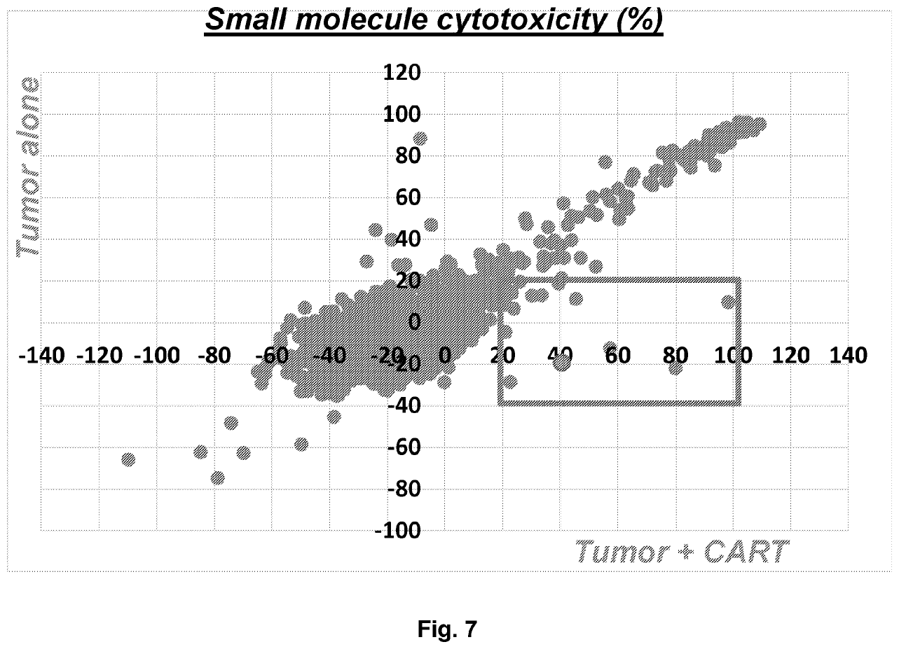

An unbiased, reliable, automated, high-throughput system was generated to test thousands of different combinations of CART and small molecules. Briefly, as shown in FIG. 6, CAR T cells were plated via an automated dispenser with or without luciferase+ tumor cells. Subsequently, 2,615 different small molecules were added (1 per well). More than half of these drugs are FDA-approved. After 48 hours, tumor luminescence was recorded and percentage tumor killing was calculated. This platform has already generated several significant hits. See FIG. 7, birinapant in grey, represented by the dot at the “40” mark on the X axis (●). Thus, small molecules enhancing CART killing can be identified.

Birinapant was the only SMAC mimetic in the library. However, the library included two drugs that also inhibit IAPs: Embelin (2,5-Dihydroxy-3-undecyl-2,5-cyclohexadiene-1,4-dione) and GDC-0152 (N-methyl-L-alanyl-(2S)-2-cyclohexylglycyl-N-(4-phenyl-1,2,3-thiadiazol-5-yl)-L-prolinamide). Neither Embelin nor GDC-0152 revealed a significant effect on CART killing activity in the screen.

### Example 2—Birinapant Enhances CART Killing of Leukemic Cell Lines

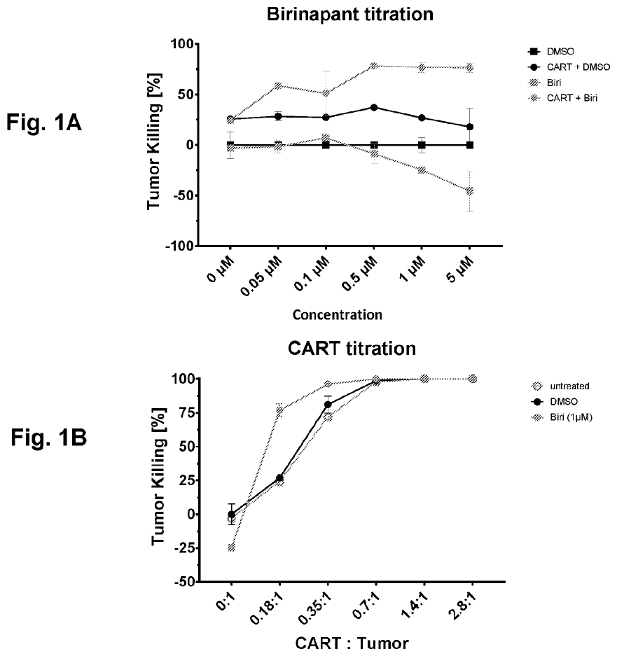

A human pre-B ALL cell line expressing endogenous CD19 and a luciferase reporter (NALM6 CBG-T2A-GFP) was cultured in the presence or absence of human T cells expressing a chimeric antigen receptor against CD19 (CTL019, KYMRIAH®) at different ratios. Cells were treated with different doses of birinapant (TL32711, Medivir) or corresponding amounts of DMSO. After 72 hours luciferin was added to the cells and luminescence was detected using a luminometer (Biotek Synergy H4). Tumor killing was calculated using the formula: (sample−tumor treated with DMSO)/(lysis control−tumor treated with DMSO). The results are shown in FIGS. 1A-1B. Negative values indicate increased tumor growth. The results show that birinapant enhances CART killing of leukemic cell lines.

### Example 3—Birinapant Enhances CART Killing of Solid Tumor Cell Lines

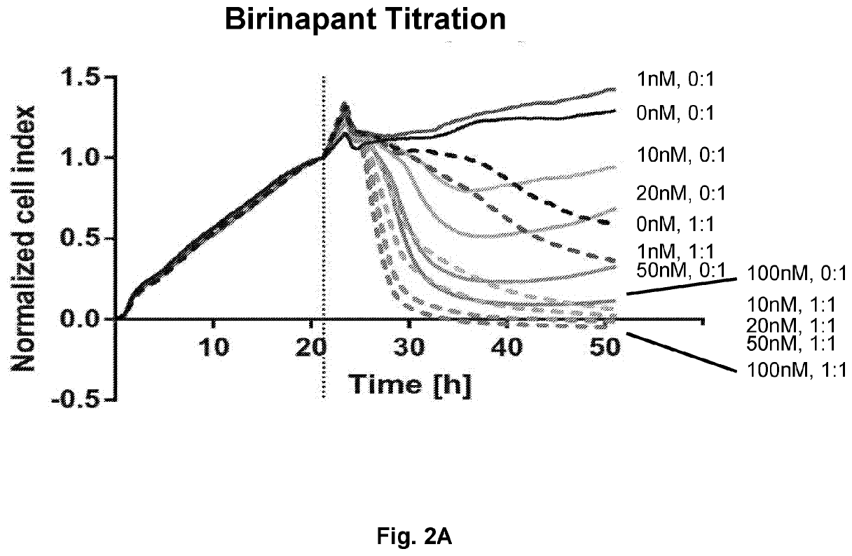

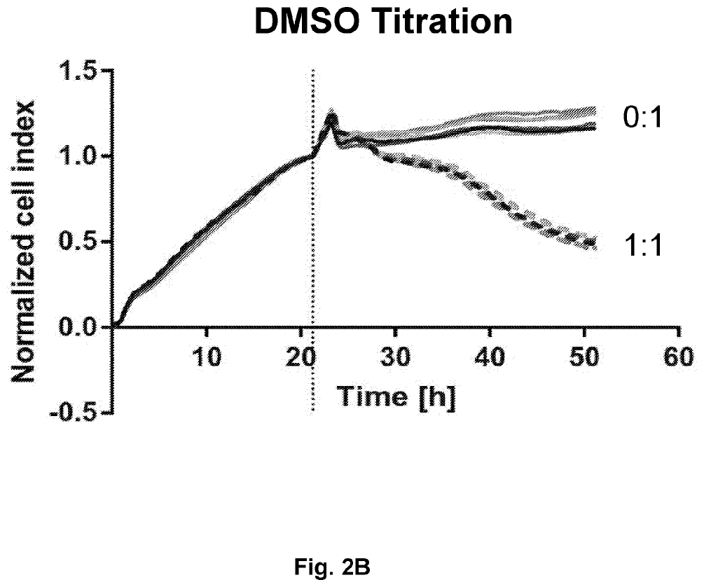

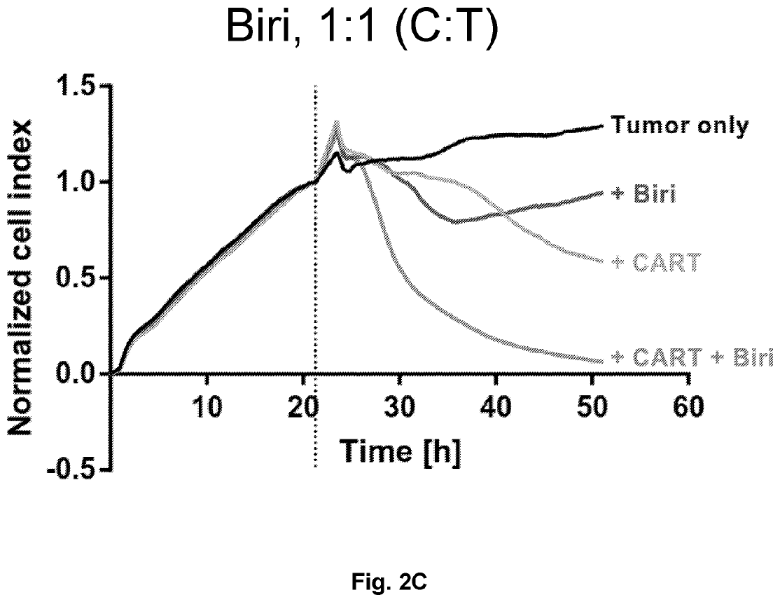

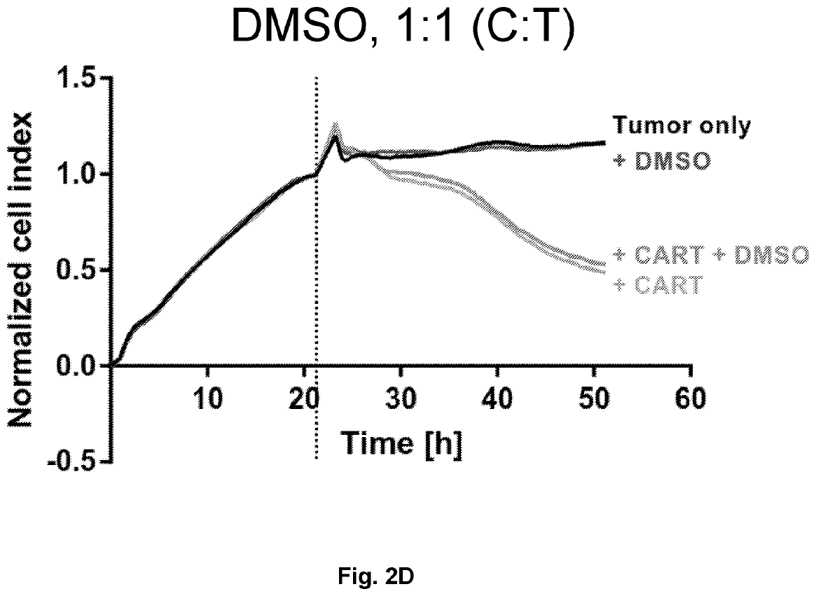

A human ovarian adenocarcinoma cell line (SKOV3) expressing endogenous HER2 was cultured in the presence or absence of human T cells expressing a chimeric antigen receptor against HER2 (CAR 4D5; Liu et al. Cancer Research 2015; (7517):3596-607, incorporated by reference herein in its entirety) at a 1:1 CART:Tumor ratio. Cells were treated with different doses of birinapant (TL32711, Medivir) or corresponding amounts of DMSO. Tumor killing was monitored in real-time using the impedance-based XCelligence® system (ACEA Biosciences, Inc). The results are shown in FIGS. 2A-2D. The results show that Birinapant enhances CART killing of solid tumor cell lines.

### Example 4—Birinapant-Enhanced CART Killing of Tumor is TNF-Dependent

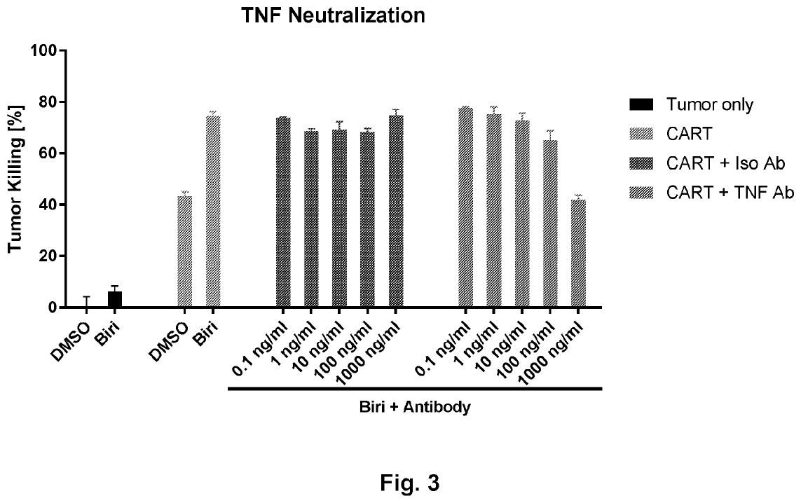

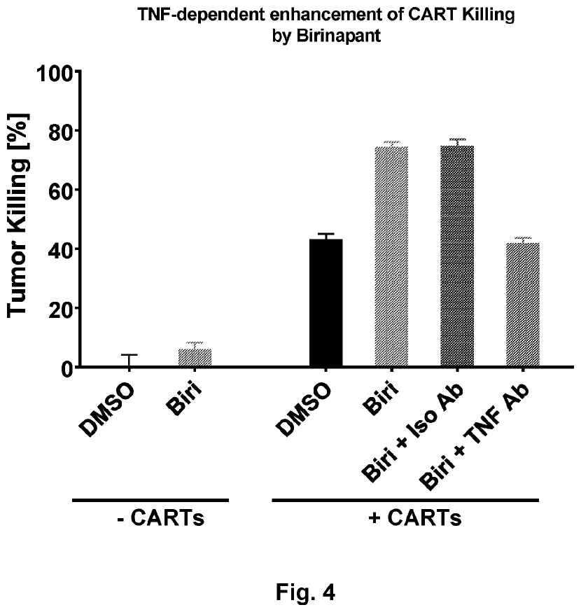

A human pre-B ALL cell line expressing endogenous CD19 and a luciferase reporter (NALM6 CBG-T2A-GFP) was cultured in the presence or absence of human T cells expressing a chimeric antigen receptor against CD19 (CTL019, KYMRIAH®). Cells were either treated with 1 μM birinapant (TL32711, Medivir), corresponding amounts of DMSO or 1 μM birinapant (TL32711, Medivir) and different amounts of isotype control or TNF antibodies. After 72 hours, luciferin was added to the cells and luminescence was detected using a luminometer (Biotek Synergy H4). Tumor killing was calculated using the formula: (sample−tumor treated with DMSO)/(lysis control−tumor treated with DMSO). The results are shown in FIGS. 3 and 4. The results show that birinapant-enhanced CART killing of tumor is TNF-dependent.

### Example 5—SMAC Mimetics Enhance CART Killing of Leukemic Cell Lines

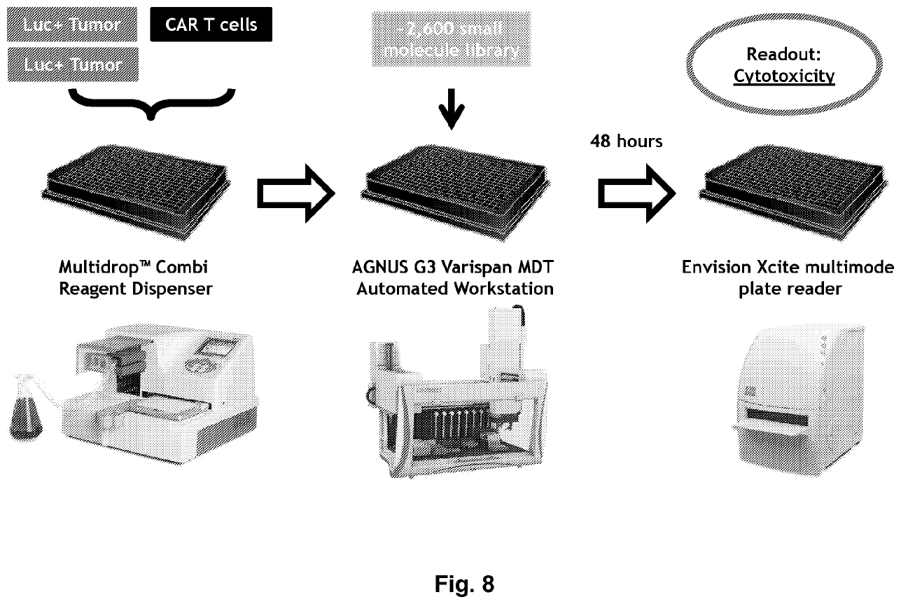

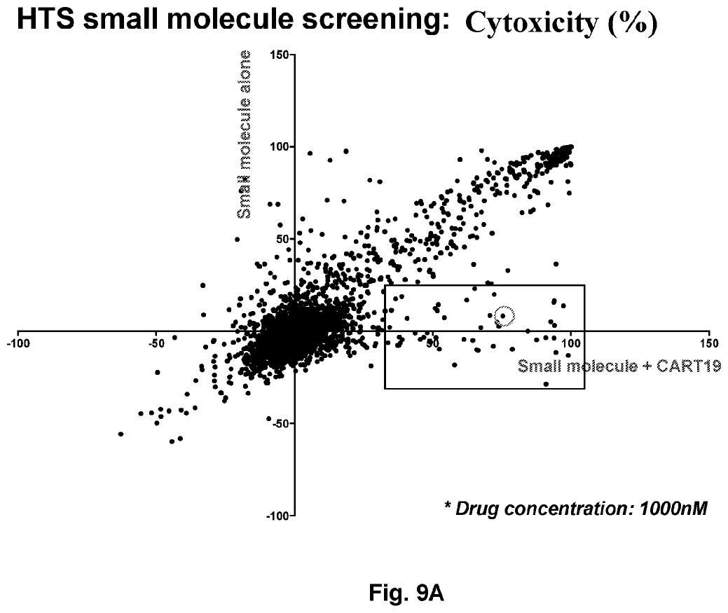

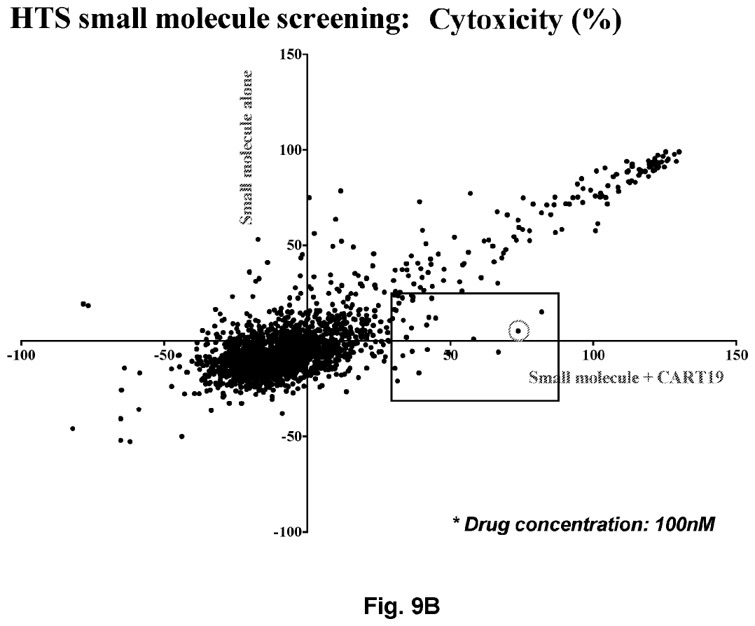

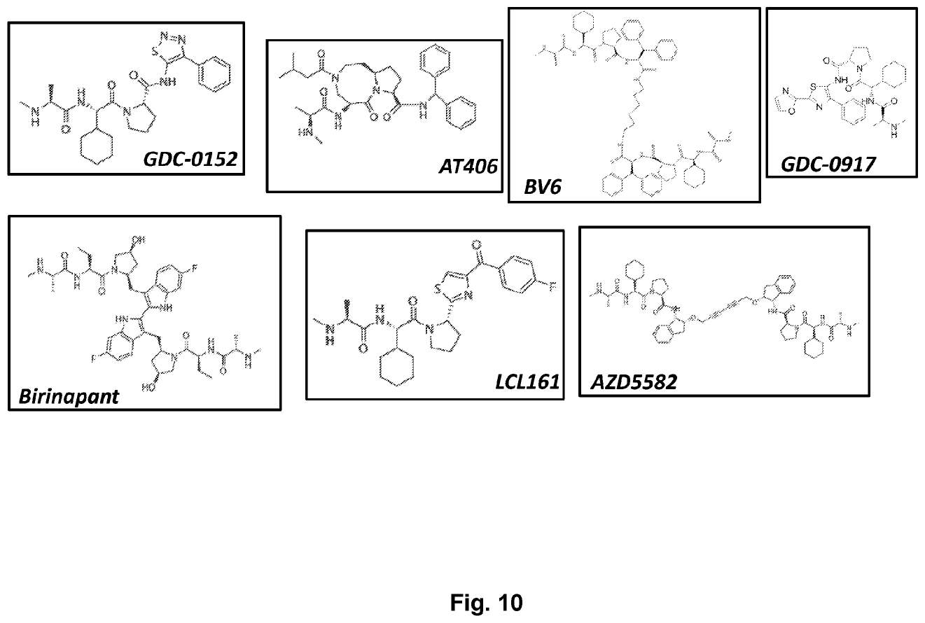

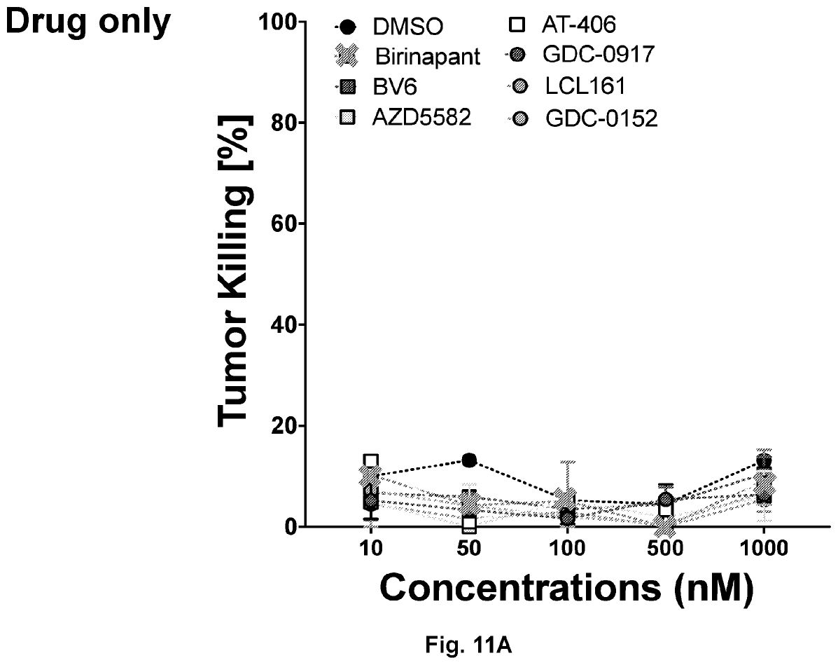

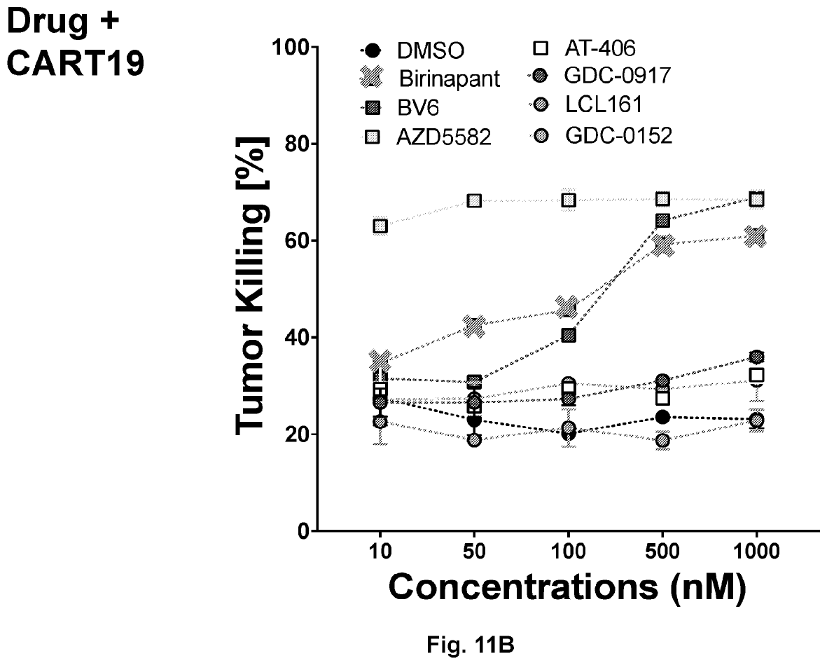

A human pre-B ALL cell line expressing endogenous CD19 and a luciferase reporter (NALM6 CBG-T2A-GFP) was cultured in the presence or absence of human T cells expressing a chimeric antigen receptor against CD19 (CTL019) at E:T ratio=0.03:1. Cells were treated with different doses of SMAC mimetics or corresponding amounts of DMSO. After 48 hours, luciferin was added to the cells and luminescence was detected using a luminometer (Biotek Synergy H4). Tumor killing was calculated using the formula: (sample−tumor treated with DMSO)/(lysis control−tumor treated with DMSO). The results are shown in FIGS. 1A-11B. The results show that SMAC mimetics enhance CART19 killing of B-cell leukemia cell lines.

### Example 6—SMAC Mimetics Enhance CART Killing of Solid Tumor Cell Lines

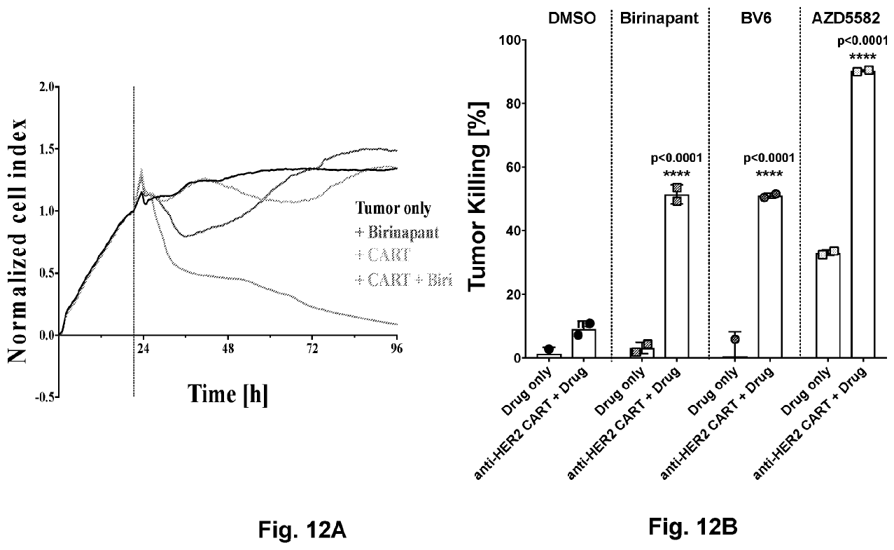

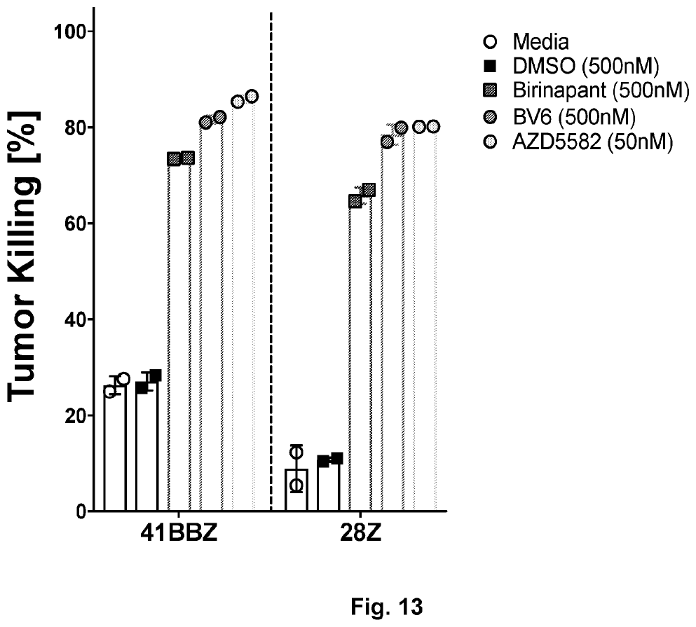

A human ovarian cell line expressing endogenous HER2 and luciferase reporter (SKOV3 CBG-T2A-GFP) was seeded at density of 10⁴ cells/100 μl of media in each well of 96 well plate and grown overnight. Next day, CAR T cells (4D5; Liu et al. Cancer Research 2015; (75)(17):3596-607, incorporated by reference herein in its entirety) were introduced at E:T ratio=0.01:1 50 nM of SMAC mimetics or corresponding amounts of DMSO. After 48 hours, luciferin was added to the cells and luminescence was detected using a luminometer (Biotek Synergy H4). Tumor killing was calculated using the formula: (sample−tumor treated with DMSO)/(lysis control−tumor treated with DMSO). A human ovarian adenocarcinoma cell line (SKOV3) expressing endogenous HER2 was cultured in the presence or absence of human T cells expressing a chimeric antigen receptor against HER2 at a 1:1 CART:Tumor ratio. Cells were treated with 50 nM of birinapant (TL32711, Medivir), BV6, AZD5582 or corresponding amounts of DMSO. Tumor killing was monitored in real-time using the impedance-based XCelligence@ system (ACEA Biosciences, Inc). The results are shown in FIGS. 12A-12B. The results show that Birinapant, BV6 and AZD5582 enhance CART killing of solid tumor cell lines.

### Example 7-SMAC Mimetics in Combination with CART Cells Induce Apoptosis of Tumor Cells

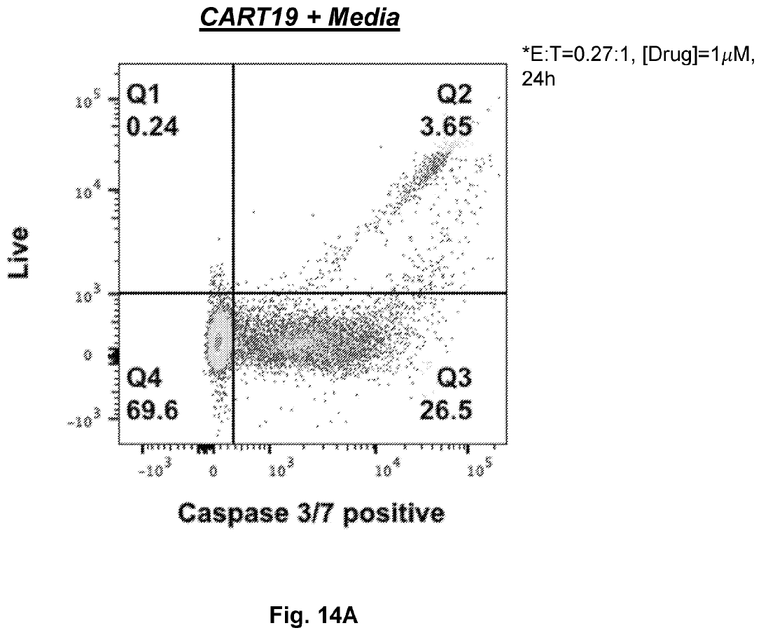

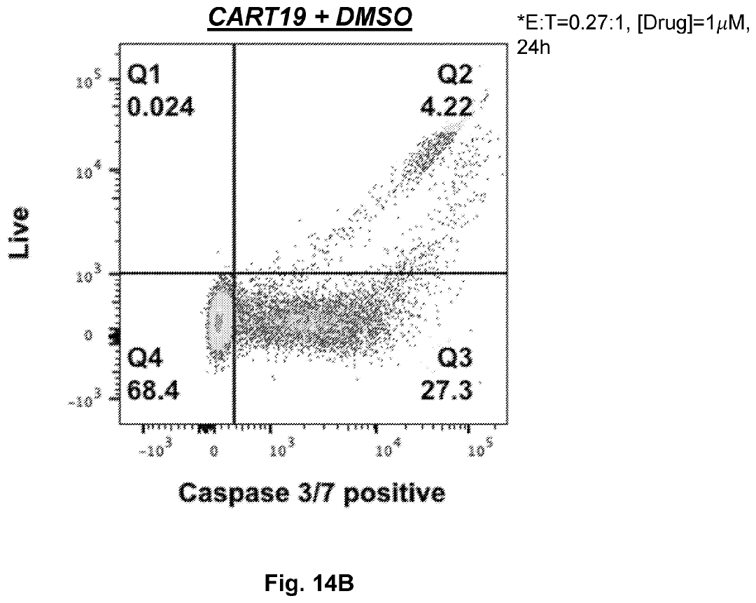

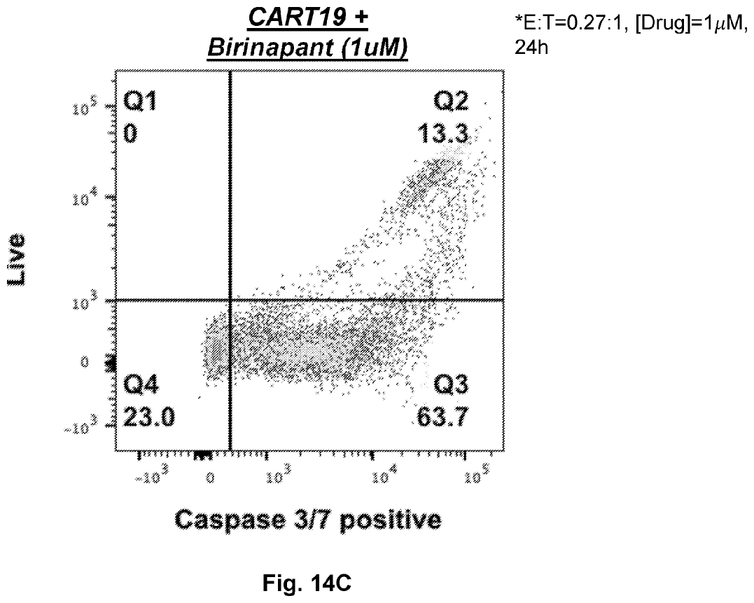

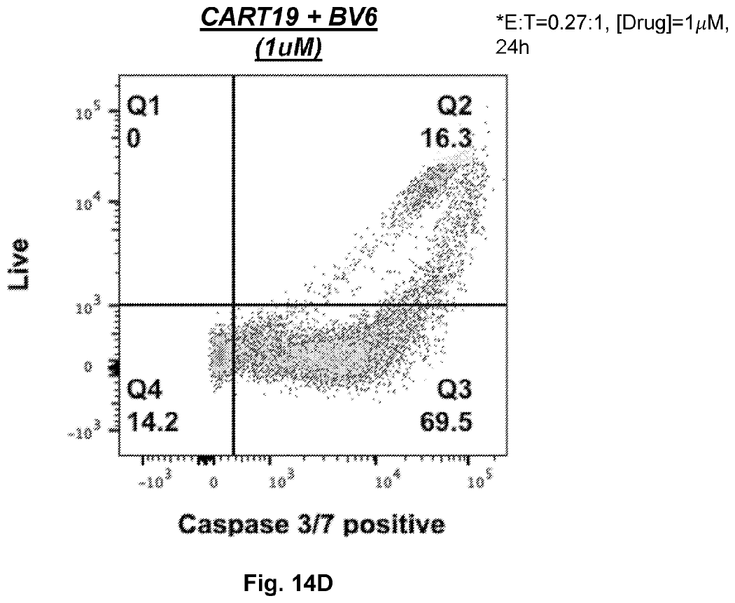

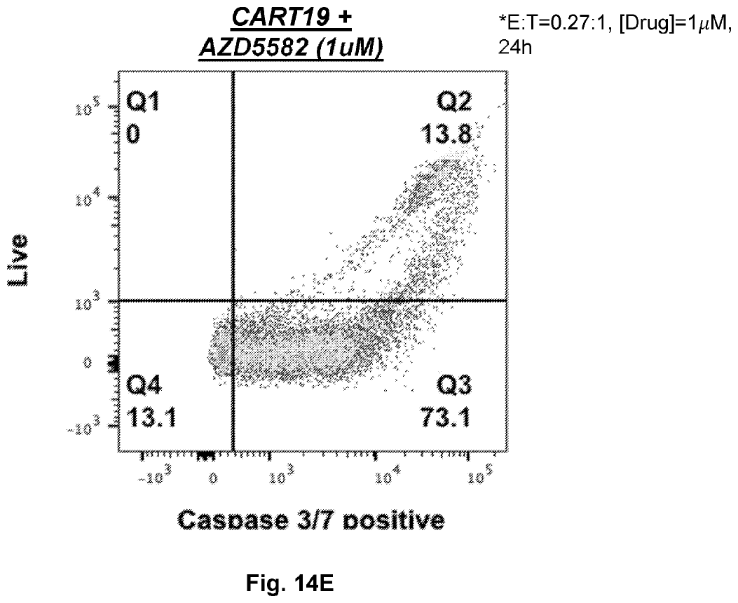

A human pre-B ALL cell line expressing endogenous CD19 and a luciferase reporter (NALM6 CBG-T2A-GFP) was cultured in the presence or absence of human T cells expressing a chimeric antigen receptor against CD19 (CTL019) at E:T ratio=0.27:1. Cells were then treated with 1 μM of SMAC mimetic or corresponding amounts of DMSO. After 24 hours, cells were harvested and measured caspase 3/7 activity via flow cytometric analysis with Invitrogen™ CellEvent™ Caspase-3/7 green ready probes Reagent following company's protocol. The results are shown in FIGS. 14A-14E. FIG. 14A shows CART19 cells alone. FIG. 14B shows CART19 cultured with DMSO. FIG. 14C shows CART19 co-cultured with Birinapant (1 μM). FIG. 14D shows CART19 co-cultured with BV6 (1 μM). FIG. 14E shows CART19 co-cultured with AZD5582 (1 μM). The results show that SMAC mimetics in combination with CART cells induce apoptosis of tumor cells.

### Example 8—Inhibition of Caspase Activity Reduces Synergy Between CART and SMAC Mimetics

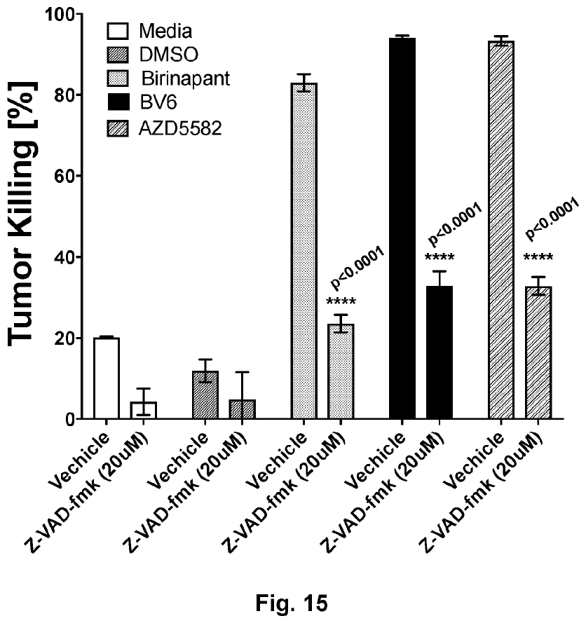

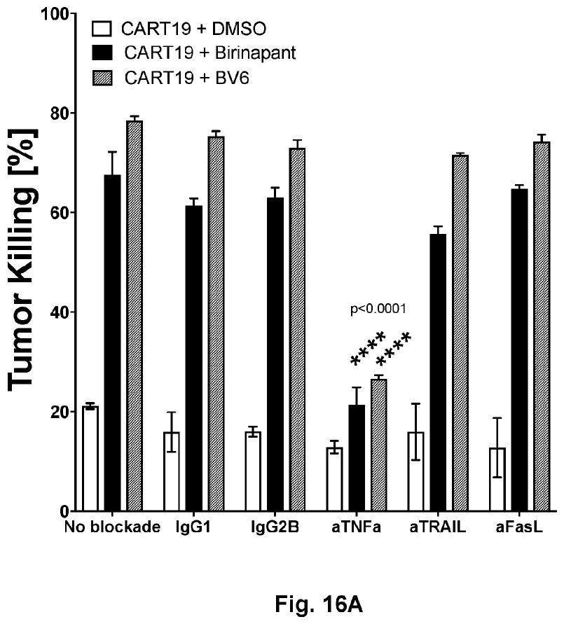

A human pre-B ALL cell line expressing endogenous CD19 and a luciferase reporter (NALM6 CBG-T2A-GFP) was cultured in the presence or absence of human T cells expressing a chimeric antigen receptor against CD19 (CTL019) at E:T ratio=0.03:1. Cells were treated with 1 μM of SMAC mimetics or corresponding amounts of DMSO in the presence or absence of pan-caspase inhibitor (Z-VAD-FMK (20 μM)). After 48 hours, luciferin was added to the cells and luminescence was detected using a luminometer (Biotek Synergy H4). Tumor killing was calculated using the formula: (sample−tumor treated with DMSO)/(lysis control−tumor treated with DMSO). CART19 cells were co-cultured with NALM6 (CBG-T2A-GFP) in the presence of DMSO (1 μM), birinapant (1 μM), BV6 (1 μM) or AZD5582 (1 μM) with or without the pan-caspase inhibitor (Z-VAD-FMK (20 μM)). The results are shown in FIG. 15. After 48 hours, total tumor killing was monitored by measuring the change of luminescence intensity, E:T ratio=0.03:1. The results show that inhibition of caspase activity reduces synergy between CART and SMAC mimetics.

### Example 9—Synergy Between CART and SMAC Mimetics is Mediated by TNFα

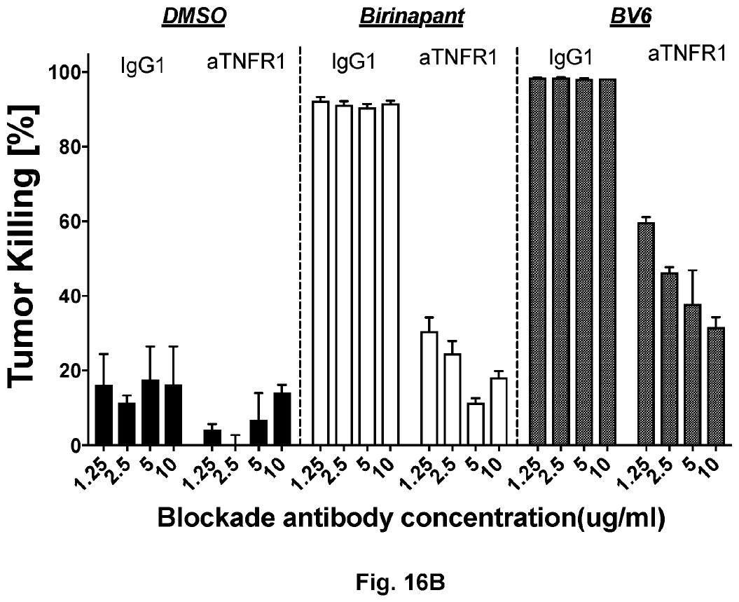

A human pre-B ALL cell line expressing endogenous CD19 and a luciferase reporter (NALM6 CBG-T2A-GFP) was cultured in the presence or absence of human T cells expressing a chimeric antigen receptor against CD19 (CTL019). Cells were either treated with 1 μM of birinapant (TL32711, Medivir), BV6 (Genentech) or corresponding amounts of DMSO. In order to block interaction of death ligands to target receptor, different amounts of isotype controls or TRAIL, TNF and FasL antibodies. Moreover, neutralizing antibodies for different amounts of isotype control and TNF receptor 1 (TNFR1) was introduced cells treated with either treated with 1 μM of birinapant (TL32711, Medivir), BV6 (Genentech) or corresponding amounts of DMSO to block binding of TNF. After 48 hours, luciferin was added to the cells and luminescence was detected using a luminometer (Biotek Synergy H4). Tumor killing was calculated using the formula: (sample−tumor treated with DMSO)/(lysis control−tumor treated with DMSO). The results are shown in FIGS. 16A-16B. FIG. 16A shows the synergy between CAR T cell and SMAC mimetics [(DMSO (1 μM), birinapant (1 μM) or BV6 (1 μM)], neutralizing antibodies for death ligands: TNFα (1 μg/ml), TRAIL (1 μg/ml), FasL (1 μg/ml). NALM6 (CBG-T2A-GFP) killing was measured by monitoring change of luminescent activity after 48 hours. E:T ratio=0.03:1. FIG. 16B shows the synergy between CAR T cell and SMAC mimetics [(DMSO (1 μM), birinapant (1 μM) or BV6 (1 μM)], neutralizing antibodies for TNF receptor 1 (TNFR1). NALM6 (CBG-T2A-GFP) killing was measured by monitoring change of luminescent activity after 48 hours. E:T ratio=0.03:1. The results show that the synergy between CART and SMAC mimetics is mediated by TNFα.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising:
a T cell genetically modified to express a CAR; and
a SMAC mimetic.

2. The composition of claim 1, wherein the CAR comprises an antigen binding domain, a transmembrane domain, and an intracellular signaling domain.

3. The composition of claim 2, wherein the intracellular signaling domain comprises a costimulatory signaling region.

4. The composition of claim 3, wherein the costimulatory signaling region comprises the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83.

5. The composition of claim 2, wherein the intracellular signaling domain comprises a CD3zeta chain.

6. The composition of claim 1, wherein the SMAC mimetic is birinapant, (methylamino)propanamide, LCL161, GDC-0917, HGS1029, AT-406, BV-6, GDC-0152, or AZD5582, or any combinations thereof, or a salt or solvate thereof.

7. The composition of claim 1, wherein the SMAC mimetic is birinapant, or a salt or solvate thereof.

8. The composition of claim 1, wherein the antigen is a tumor antigen.

9. The composition of claim 1, wherein the tumor antigen is selected from the group consisting of CD19, CD20, CD22, BCMA, ROR1, Mesothelin, CD33/IL3Ra, c-Met, PSMA, Glycolipid F77, EGFRvIII, GD-2, NY-ESO-1 TCR, MAGE A3 TCR and HER-2.

10. The composition of claim 1, further comprising a pharmaceutically acceptable carrier or adjuvant.

11. A method for treating a patient having a disease, a disorder or a condition associated with an elevated expression of an antigen, the method comprising administering to the patient an effective amount of the composition of claim 1.

12. A method for treating a patient having a disease, a disorder or a condition associated with an elevated expression of an antigen, the method comprising administering to the patient an effective amount of:
a T cell genetically modified to express a CAR; and
a SMAC mimetic.

13. The method of claim 12, wherein the CAR comprises an antigen binding domain, a transmembrane domain, and an intracellular signaling domain.

14. The method of claim 13, wherein the intracellular signaling domain comprises a costimulatory signaling region.

15. The method of claim 14, wherein the costimulatory signaling region comprises the intracellular domain of a costimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83.

16. The method of claim 13, wherein the intracellular signaling domain comprises a CD3zeta chain.

17. The method of claim 12, wherein the SMAC mimetic is selected from the group consisting of AZD5582, birinapant, LCL161, GDC-0152, GDC-0917, HGS1029, AT-406, or BV-6, any salt or solvate thereof, and any combinations thereof.

18. The method of claim 17, wherein the SMAC mimetic is birinapant, or a salt or solvate thereof.

19. The method of claim 12, wherein the T cell genetically modified to express a CAR and the SMAC mimetic are administered to the patient simultaneously or sequentially.

20. The method of claim 12, wherein the antigen is a tumor antigen.

21. The method of claim 20, wherein the tumor antigen is selected from the group consisting of CD19, CD20, CD22, BCMA, ROR1, Mesothelin, CD33/IL3Ra, c-Met, PSMA, Glycolipid F77, EGFRvIII, GD-2, NY-ESO-1 TCR, MAGE A3 TCR and HER-2.

22. The method of claim 12, wherein the T cell genetically modified to express a CAR and/or the SMAC mimetic further comprises a pharmaceutically acceptable carrier or adjuvant.
